(19) United States
(12) Patent Application Publication
(10) Pub. No.: US 2006/0009430 A1
(43) Pub. Date: Jan. 12, 2006

(54) Composition for the prevention and treatment of the detrimental effects of dihydrotestosterone

(76) Inventor: Gregory J. Kelly, Glastonbury, CT (US)

Correspondence Address: MCCARTER & ENGLISH LLP;CITYPLACE I, 185 ASYLUM STREET, HARTFORD, CT, 06103, US

(21) Appl. No.: 11/171,678

(22) Filed: Jun. 30, 2005

Related U.S. Application Data
(60) Provisional application No. 60/585,189, filed on Jul. 1, 2004.

(51) Int. Cl. A61K 31/57

(52) U.S. Cl. 514/171

(57) ABSTRACT

The composition of the invention relates generally to a nutritional supplement, which is capable of ameliorating the biologically detrimental effects of dihydrotestosterone (DHT). The present composition comprises a combination of natural plant-derived extracts, and minerals including Saw palmetto berry extract; pumpkin seed (Curcubito pepo) extract; beta-sitosterol; quercetin; Polygonum multiflorum root extract; a lycopene, for example from tomato extract; isoflavones, for example from Pueraria lobata root extract, and soybeans; zinc; selenium or any combination thereof. The ingredients are combined into any suitable form to be administered, and consumed by an individual for the prevention or treatment of hair loss, or prostatic hyperplasia or both.

### CROSS-REFERENCE TO RELATED APPLICATIONS

Under 35 U.S.C. § 119(e) this application claims the benefit of U.S. Provisional Application No. 60/585,189 filed Jul. 1, 2004, and is hereby incorporated by reference in its entirety.

### FIELD OF THE INVENTION

The present invention relates generally to a composition, which is capable of ameliorating the biologically detrimental effects of 5-alpha-dihydrotestosterone (DHT), for example hair loss (i.e. androgenic alopecia), and prostatic hyperplasia. In particular the present composition relates to a combination of natural plant-derived ingredients, and optionally including at least one additional component, for example a mineral component, a pharmaceutically acceptable carrier, excipient or preservative to be administered to an individual for the treatment of prostate hyperplasia, androgenic alopecia, or both.

### BACKGROUND

Androgenic hormones, such as testosterone, are critical for the normal development and maturation of primary and secondary male sexual characteristics. Androgens exert their effects by binding to intracellular androgen receptor (AR) proteins, which then translocate to the nucleus of a cell, and bind to genomic deoxyribonucleic acid (DNA) to initiate transcription of specific genes related to growth and development. Androgen receptors are present in the cells of several organs including the prostate, the skin, and hair follicles where the ARs mediate normal development by androgenic hormones. While testosterone is the primary ligand for ARs during pre-adult development, incompletely understood effects of aging or genetic predisposition causes other forms of androgenic hormones to accumulate in some organs such as the prostate, skin, and hair follicles, and compete for ARs, which can lead to detrimental health-effects, for example prostatic hyperplasia, and alopecia (i.e. hair loss).

Alopecia can start as early as the teens in genetically susceptible males, and it has been estimated to be present in about a third of males by age 30, and nearly half of males by age 50. Risk factors for alopecia that have been identified include psychological illness, and exposure to chemotherapeutic agents, and radiation. However, at least one mechanism of hair loss, androgenic alopecia, commonly referred to as male-pattern baldness, is believed to result from the production of the testosterone analog, 5-alpha-dihydrotestosterone (DHT).

Testosterone is converted into DHT in the cells of the hair follicles by the action of the enzyme, 5-alpha-reductase. The detrimental effects of DHT are likely due to fact that it is a more potent agonist of AR activity than testosterone. Furthermore, follicular hair loss has been correlated with higher relative expression of the 5-alpha-reductase gene, which likely results in increased DHT formation in the scalp, and the resulting hair loss observed in male-pattern baldness.

In addition, increased production of DHT in the prostate has been associated with the progression of prostate cancer. Approximately 70% of males in the U.S. over the age of 50 have pathological evidence of prostate hyperplasia, and prostate cancer is the second leading cause of cancer death in men in the United States, exceeded only by lung cancer. The American Cancer Society estimates that 29,900 men in the United States will die of prostate cancer during 2004.

Excessive AR activity has also been reported to cause acne, which is the most common skin disorder treated by physicians, as well as hirsutism in women.

One currently available treatment for androgenic alopecia includes finasteride, a chemical inhibitor of the 5-alpha-reductase type II enzyme. An additional treatment option includes Minoxidil, whose precise mechanisms of effect are unclear. However, these treatments are unfavorable for several reasons including cost, immunogenicity, and undesirable side effects.

One caveat of prostate hyperplasia, and alopecia or male-pattern baldness is the required presence of both 5-alpha-reductase activity, and the androgen receptor. As a consequence, inhibiting the activity of 5-alpha-reductase, or inhibiting the binding of DHT to the ARs or both will effectively prevent or ameliorate prostatic hyperplasia or hair loss in follicles suffering from androgenic alopecia.

Chizick et al., U.S. Pat. No. 5,972,345 shows a natural formulation for the treatment of hair loss that requires Saw palmetto, African Pygeum extract, and stinging nettle extract. However, the '345 patent does not teach or suggest use of selenium, lycopenes, pumpkin seed extract, quercetin, or Kudzu extract for the prevention and treatment of the detrimental effects of DHT.

Catalfo, U.S. Pat. No. 6,596,266 shows a composition for the treatment of hair loss that requires Saw palmetto, stinging nettle root, as well as Minoxidil. The '266 patent does not teach or suggest using selenium, lycopenes, pumpkin seed extract, quercetin, or Kudzu extract for the prevention and treatment of alopecia, and prostatic hyperplasia related to DHT.

Therefore there exists a need for a natural dietary supplement that is better tolerated than synthetic pharmaceuticals, and is uniquely formulated with natural ingredients that are beneficial in treating the detrimental effects of DHT including alopecia, and prostatic hyperplasia.

### SUMMARY OF THE INVENTION

This application claims the benefit of U.S. Provisional Application No. 60/585,189 filed Jul. 1, 2004, and is hereby incorporated by reference in its entirety.

In one aspect, the present invention relates to a novel composition useful for administration to an individual for the prevention and treatment of the detrimental effects of DHT, for example prostate hyperplasia, alopecia or both. The composition is comprised of Saw palmetto berry extract, pumpkin seed (Curcubito pepo) extract, and at least one of beta-sitosterol, and quercetin. In another aspect the composition optionally comprises at least one of Polygonum multiflorum root extract; Kudzu root extract; lycopene, for example from tomato extract; isoflavones, for example, from Pueraria lobata root standardized extract, and soybeans; zinc; selenium or any combination thereof.

In another aspect, the invention relates to providing methods of administering a novel composition to a patient for the prevention or therapeutic treatment of the detrimental effects of DHT. In certain aspects, the method comprises administering an effective amount of the composition of the invention to a person in need thereof for the treatment of the detrimental effects of DHT.

Among the objects of the invention is to provide a composition administered in any pharmaceutically acceptable carrier, for example: a gel, lotion, shampoo, spray, powder, pill, tablet, emulsion, liquid, salt, paste, jelly, aerosol, ointment, capsule, gel cap, controlled release tablet or capsule, microspheres, enteric coated tablets or capsules, subdermal implant, dermal patch or any other suitable form known to those of skill in the art. In addition the composition of the invention may be administered by any suitable route, for example, dermal, oral, nasal, and intravenous or any combination thereof.

In certain embodiments the composition of the invention may optionally be combined with one or more pharmaceutically acceptable excipients. In certain aspects excipients are employed to increase efficacy, bioavailability, absorption, and shelf-life or to create a suitable form, taste or consistency for administration to an individual. Examples of excipients that can be used with the composition of the invention include, lipids, phosphates, phospholipids, triglycerides, fats, oils, acids, folic acid, bases, emulsifiers, surfactants, saccharides, sugars, carbohydrates, chelators, sweeteners, alcohols, glycols, antioxidants, gelatin, glycerin, soybean oil, beeswax, lecithin, corn oil, vitamins, salts, and the like. The above-mentioned excipients are offered by way of non-limiting example. As will be understood by one of ordinary skill in the art, the ingredients disclosed herein may be varied in any number of ways, and ratios in order to achieve the composition of the invention without departing from the general scope of the invention.

One of the advantages of the present invention is that the composition is comprised of natural ingredients, which are safe, and that produce effects that are beneficial in treating the detrimental effects of DHT, and other physical symptoms, and moreover, the combination of these substances in the proportions discussed below results in a composition that is uniquely beneficial for preventing or treating the detrimental effects of DHT, including alopecia and prostate hyperplasia. Other benefits and advantages will be appreciated in view of the detailed description and examples of the preferred embodiments provided herein.

### DESCRIPTION OF THE PREFERRED EMBODIMENTS

This application claims the benefit of U.S. Provisional Application No. 60/585,189 filed Jul. 1, 2004, and is hereby incorporated by reference in its entirety.

The novel composition of the present invention and its method of use are directed to the treatment and prevention of the biologically detrimental effects of 5-alpha-dihydrotestosterone (DHT). In particular the invention is directed to a novel composition, and method that may be used for the treatment and prevention of androgenic alopecia (i.e. male pattern baldness), prostatic hyperplasia or both. In a particularly preferred embodiment, the composition of the present invention comprises a combination of plant-derived extracts, and optionally includes a mineral component, pharmaceutically acceptable excipients or both.

In a preferred embodiment of the present invention the composition comprises Saw palmetto berry extract, pumpkin seed extract (Curcubito pepo), and at least one of beta sitosterol, and quercetin. In another preferred embodiment the composition described above also contains at least one additional component selected from the group consisting of Polygonum multiflorum, Kudzu root extract, lycopenes, tomato extract, isoflavones, Pueraria lobata extract, soybean, zinc, selenium and any combination thereof. Each of the ingredients or components produces effects that are beneficial in preventing or treating the detrimental effects of dihydrotestosterone, and moreover, the combination of these substances in the proportions discussed below results in a composition that is uniquely beneficial for ameliorating the harmful effects of DHT.

Saw palmetto is believed to inhibit the actions of testosterone by inhibiting its conversion to DHT, and by blocking DHT binding to ARs. Among the main constituents of saw palmetto berry extract are fatty acids and sterols. The fatty acids are purported to be responsible for testosterone-inhibiting effects within the prostate. A double-blind clinical study was performed with 110 patients receiving saw palmetto or placebo for 30 days. Based on objective measurements of the symptoms of benign prostate hyperplasia (BPH) and a physician's assessment, the extract was superior to placebo. The extract has also been shown to have anti-inflammatory effects. Double-blind human clinical studies have shown that 320 mg per day of saw palmetto extract was sufficient to reduce symptoms associated with BPH. The German Commission E (which is a panel of medical and pharmacy experts within the German government), has approved the saw palmetto for use in early stage BPH at the dosage of 320 mg per day of the fatty acids.

The mechanism of action of the sterols is not well understood, although they are present in other herbs used in treating symptoms of prostate hyperplasia. Saw palmetto may also improve general bladder and urinary tract health, hormonal balance, and respiratory health. In one embodiment the composition contains from about 0.1% to about 80% by weight of Saw palmetto berry extract. Preferably, the composition contains from about 10% to about 70% by weight of Saw palmetto berry extract.

Beta-sitosterol is plant sterol found in almost all plants. It is one of the main subcomponents of a group of plant sterols known as phytosterols. It is white in color and waxy in nature. It has a chemical structure that is very similar to cholesterol. High levels of beta-sitosterol are found in rice bran, wheat germ, corn oils, and soybeans. Studies have indicated that beta-sitosterol is effective in preventing and treating prostate problems such as prostatic hyperplasia. In addition, beta-sitosterol is an effective option in lowering cholesterol, increasing immunity, and reducing inflammation. In one embodiment the composition contains from about 0% to about 80% by weight of beta-sitosterol. Preferably, the composition contains from about 0% to about 70% by weight of beta-sitosterol.

Pumpkin seeds are a natural source of B-vitamins, zinc, alpha-linolenic acid, amino acids and phytosterols, which support prostate and bladder health, enhance immune function, and promote cardiovascular health. Research has shown that pumpkin seed oil can reduce the symptoms associated with BPH. The dosage usually used in the studies is 160 mg of pumpkin seed oil administered three times per day. In one embodiment the composition contains from about 0.1% to about 80% by weight of pumpkin seed extract. Preferably, the composition contains from about 10% to about 70% by weight of pumpkin seed extract.

Phytoestrogens, such as flavonoids and isoflavonoids, are polyphenolic compounds commonly found in many plants and widely used for their medicinal value. These compounds may be both agonists and antagonists of estrogen receptors in humans. It has been proposed that these estrogenic compounds may influence prostate cancer cell growth. In vivo and in vitro studies have shown that some phytoestrogens influence not only steroid hormone metabolism and function but also intracellular enzymes, growth factor action, protein synthesis, malignant cell proliferation and angiogenesis, thus making them strong candidates for therapeutic usage. Examples of phytoestrogens, which have similar chemical structure to that of estrogen, include quercetin, beta-sitosterol, and the isoflavonoids in soy and kudzu, such as genistein and daidzein.

Quercetin, a phenylbenzopyrone, belongs to a class of water-soluble plant polyphenolic pigments called flavonoids, which have well known anti-oxidant properties. Preliminary studies indicate that quercetin may inhibit the proliferation of androgen-independent human prostatic tumor cells, and has been shown to result in cell growth cycle arrest, and to induce apoptosis in some cancer cells. In rats, quercetin, in combination with finasteride, has been show to reduce prostate weight. Whether quercetin therapy alone benefits those with benign prostate hyperplasia is not clearly known at this time. Quercetin also inhibits lipid peroxidation, acts as an antihistamine, and has anti-inflammatory properties. These antioxidant and anti-inflammatory activities likely account for quercetin's vasoprotective activity.

Quercetin may help treat or prevent prostate cancer by blocking male hormones that encourage the growth of prostate cancer cells, and may alleviate symptoms associated with an inflamed prostate (prostatitis). In addition to promoting cardiovascular health, and inhibiting cancer growth, quercetin may also protect against the development of such diabetic complications as cataracts, retinopathy, neuropathy, and nephropathy. In one embodiment the composition contains from about 0% to about 80% by weight of quercetin. Preferably, the composition contains from about 0% to about 70% by weight of quercetin.

Polygonum multiflorum, was traditionally used by the Chinese to promote longevity, this herb is rich in flavonoids and is primarily used to restore vigor, strengthen the cardio-vascular system, enhance the endocrine system and immune functions, and tone the liver and kidneys. It is good in applications for chronic fatigue or degenerative conditions. In one embodiment the composition contains from about 0.1% to about 50% by weight of Polygonum multiflorum. In a preferred embodiment the composition contains from about 15% to about 35% by weight of Polygonum multiflorum.

Isoflavone rich products may protect against enlargement of the male prostate gland. Studies show isoflavones slowed prostate cancer growth and caused prostate cancer cells to die. Isoflavones act against cancer cells in a way similar to many common cancer-treating drugs. Isoflavones help in the preservation of bone and fight osteoporosis. Unlike estrogen, which helps prevent the destruction of bone, evidence suggests that isoflavones may also assist in creating new bone. Isoflavones act against cancer cells in a way similar to many common cancer-treating drugs. Population-based studies show a strong association between consumption of isoflavones and a reduced risk of breast and endometrial cancer. Isoflavones can be found in many foods but the most abundant source of isoflavones is the soy bean. Most of the benefits which are attributed to soy are produced by isoflavones.

Soy bean is a plant cultivated as foodstuff whose health properties have recently been discovered. Thorough studies have revealed that the consumption of the soy beans or soy foods containing isoflavones have favorable effects on people's health. A recent study has demonstrated that isoflavones have potent antioxidant properties, comparable to that of vitamin E. The anti-oxidant powers of isoflavones can reduce the long-term risk of cancer by preventing free radical damage to DNA. Genistein is the most potent antioxidant among the soy isoflavones, followed by daidzein. Isoflavones represent one of the classes of the so-called “phytoestrogens.” These bioactive non-nutrients are strikingly similar in chemical structure to estradiol, the main female hormone. Indeed, one can superimpose almost exactly the structures of estradiol and isoflavones so they become indistinguishable, and therefore they fit beautifully into the pocket representing the binding domain of the estrogen receptor. It is therefore not surprising that isoflavones share many of the properties of endogenous estrogens.

Soy contains many isoflavones, but the most beneficial are genistein and daidzein. Soy isoflavones have the ability to bind to estrogen receptors and partly block the effects of estrogen. They also bind to testosterone receptors in a similar manner, suggesting a potential use in prostate cancer. Genistein has other properties as well, such as inhibiting angiogenesis and inhibiting enzymes, including tyrosine kinase, which is directly involved in cancer cell growth and regulation..

Kudzu root (Pueraria lobata) extract was recommended for allergies, migraine headaches, measles eruptions in children, and diarrhea. It was also used as a treatment for angina pectoris. The primary chemical constituents of Kudzu include starch, isoflavonoids (i.e. plant phytoestrogens), puerarin, daidzein, and phytoestrogens. Kudzu root is high in isoflavones, such as daidzein, as well as isoflavone glycosides, such as daidzein and puerarin. Traditional Asian healers, however, have long valued kudzu's root and flowers for treating colds, flu, high blood pressure, chest pain, allergies, and a host of other ailments. And research indicates that a compound in the root (an isoflavone called puerarin) may also increase blood flow to the heart and brain--which may explain certain traditional uses.

In one embodiment the composition contains from about 0.1% to about 20% by weight of an isoflavone, for example soybean or Kudzu root extract. In a preferred embodiment the composition contains from about 1% to about 10% by weight of an isoflavone, for example soybean or Kudzu root extract.

Lycopene is an open-chain unsaturated carotenoid that imparts red color to tomatoes, and is a proven antioxidant. Antioxidants neutralize free radicals, which may damage the body's cells. Research shows that lycopene in tomatoes can be absorbed more efficiently by the body if processed into juice, sauce, paste and ketchup. The chemical form of lycopene found in tomatoes is converted by the temperature changes involved in processing to make it more easily absorbed by the body. In the body, lycopene is deposited in the liver, lungs, prostate gland, colon and skin. Its concentration in body tissues tends to be higher than all other carotenoids. Epidemiological studies have shown that high intake of lycopene-containing vegetables is inversely associated with the incidence of certain types of cancer. For example, habitual intake of tomato products has been inversely associated with the risk of cancer of the digestive tract among Italians. In one six-year study by Harvard Medical School and Harvard School of Public Health, only tomato products (which contain large quantities of lycopene) showed a measurable relationship to reduce prostate cancer risk. As consumption of tomato products increased, levels of lycopene in the blood increased, and the risk for prostate cancer decreased. The study also showed that the heat processing of tomatoes and tomato products increases lycopene's bioavailability.

Lycopene has demonstrated some mechanisms of action against cancer by, for example, reducing the rate of G1 cell cycle progression in cancer cells. Additionally, in a study performed at the Barbara Ann Karmanos Cancer Institute in Detroit, 30 men with localized prostate cancer scheduled to undergo prostate removal were orally administered either 15 mg of lycopene, twice per day or no intervention. After 3 weeks, the prostate gland was removed and biopsied. The biopsy from the lycopene group showed significant reduction in PSA, smaller tumors, reduced metastasis, and signs of regression and reduced malignancy.

Ongoing preliminary research suggests that lycopene is associated with reduced risk of macular degenerative disease, serum lipid oxidation and cancers of the lung, bladder, cervix and skin. Studies are underway to investigate other potential benefits of lycopene. These studies will focus on lycopene's possible role in the fight against cancers of the digestive tract, breast and prostate cancer. In one embodiment the composition contains from about 0.1% to about 10% by weight of a lycopene, for example a tomato extract. In another embodiment the composition contains from about 0.5% to about 1% by weight of a lycopene, for example a tomato extract.

Zinc is an essential mineral that is found in almost every cell. It stimulates the activity of over 200 enzymes. Zinc supports normal growth and development, a healthy immune system, wound healing, helps maintain your sense of taste and smell, and is needed for DNA synthesis. Zinc deficiency most often occurs when zinc intake is inadequate or poorly absorbed, when there are increased losses of zinc from the body, or when the body's requirement for zinc increases. Signs of zinc deficiency include growth retardation, hair loss, diarrhea, delayed sexual maturation and impotence, eye and skin lesions, and loss of appetite. There is also evidence that weight loss, delayed healing of wounds, taste abnormalities, and mental lethargy can occur.

Highly concentrated in red and white blood cells, zinc contributes to the proper functioning of a number of hormones, including growth and sex hormones. For example, zinc has been shown to support healthy levels of the hormone dihydrotestosterone (DHT). Zinc arginate has been shown in animal studies to increase zinc levels in the male reproductive organs more effectively than other forms of zinc. Zinc also offers powerful support for the immune system, antioxidant metabolism, energy metabolism, and synthesis of DNA and RNA. Although severe zinc deficiencies are rare, research indicates that many Americans may not get the zinc they need. In one embodiment the composition contains from about 0. 1% to about 20% by weight of zinc arginate. In one embodiment the composition contains from about 1% to about 10% by weight of zinc arginate.

Selenium is an essential trace element for humans and other animals. Selenium is incorporated into molecules of an enzyme called glutathione peroxidase. This vital enzyme protects red blood cells and cell membranes against undesirable reactions with soluble peroxides. The health of blood vessels is important to prostate health tissue as well as hair follicles because these vessels supply tissues with oxygen and nutrients. In addition, Selenium is a free-radical scavenging anti-oxidant, which protects various body systems from the damages of lipid peroxidation. Selenium has been shown to reduce oxidative damage and platelet aggregation in the blood vessels, which promotes prostacyclin activity.

Selenium deficiencies are associated with increased risk of cardiomyopathy and myocardial deaths, cancer, impaired immune function, rheumatoid arthritis, and impaired fetal development. It is also known that Selenium exerts a chemoprotective effect against certain cancers, particularly prostate cancer. What remains elusive is the mechanism by which it is able to do this.

In 1996, a cancer research study with selenium indicated that cancer mortality and morbidity was reduced by approximately 50 percent. In addition, the risk of lung, prostate and colon cancer was reduced significantly. Another study was performed evaluating selenium supplementation related to prostate cancer incidence. There were 974 men that had a history of squamous or basal cell skin carcinoma. They were given either selenium at 200 μg daily or a placebo for 4.5 years and then followed up for another 6.5 years. There was a significant 63 percent reduction in the incidence of the secondary endpoint of prostate cancer. The selenium group had only 13 that developed prostate cancer, whereas the placebo group had 35. There were no changes in skin cancer, but significant benefits in total, colorectal and lung cancer. Another study found that men consuming the most dietary selenium (assessed indirectly by measuring toenail selenium levels) developed 65% fewer cases of advanced prostate cancer than did men with the lowest levels of selenium intake.

Selenomethionine contains selenium in a molecularly integrated form and is therefore directly incorporated into the proteins in the body in place of the amino acid, methionine. It is reported that the replacement of methionine by selenomethionine in the protein structure does not induce any functional changes in the protein molecule. In fact, selenium in the protein structure protects DNA from oxidation more efficiently than the original sulfur in methionine.

Selenomethionine administered in the form of selenomethionine containing yeast was found to be effective in reducing the risk of cancer. Due to its inherent safety, and its scientific recognition as the most bioavailable form of selenium, selenomethionine and yeast containing selenomethionine seem to be the ideal forms of selenium for use in nutritional supplements and foods. In one embodiment the composition contains from about 0.01% to about 5% by weight of selenium, for example in the form of selenomethionine. In one embodiment the composition contains from about 0.015% to about 2% by weight of selenium, for example in the form of selenomethionine.

An aspect of any of the above-described embodiments of the composition of the present invention includes a suitable means for administering the composition to a person. Potential means of administering the composition include, for example, a gel, lotion, shampoo, spray, powder, pill, tablet, emulsion, liquid, salts, pastes, jellies, aerosols, ointments, capsules, gel caps, controlled release capsules or any other suitable form that will be obvious to one of ordinary skill in the art. In another embodiment of the composition of the present invention includes the use of one or more inert ingredients such as a pharmaceutically acceptable excipient or carrier to create a suitable form or consistency for administration to patients, for example gelatin, glycerin, soybean oil, beeswax, lecithin, or corn oil. In another aspect of this embodiment the composition of the invention may optionally contain one or more preservatives to prolong product shelf-life. As one of ordinary skill in the art will recognize any of the above-described inert ingredients may or may not be added in any suitable combination without limiting the scope of the present invention.

A further object of the present invention is to provide a kit comprising a suitable container, the composition of the invention in a pharmaceutically acceptable form disposed therein, and instructions for its use.

Additional objects and advantages of the present invention will be appreciated by one of ordinary skill in the art in light of the current description and examples of the preferred embodiments, and are expressly included within the scope of the present invention.

It is understood that the examples and embodiments described herein are for illustrative purposes only and that various modifications or changes in light thereof will be suggested to persons skilled in the art and are included within the spirit and purview of this application and are considered within the scope of the appended claims. All publications, patents, and patent applications cited herein are hereby incorporated by reference in their entirety for all purposes.

In examples of the preferred embodiments, the useful and advantageous administration of a composition for inhibiting the detrimental effects of DHT is disclosed. The following detailed examples are given by way of example of the preferred embodiments, and are in no way considered to be limiting to the invention. For example, the relative quantities of the ingredients may be varied to optimize the desired effects, additional ingredients may be added, and/or similar ingredients may be substituted for one or more of the ingredients described. Additional advantageous features and functionalities associated with the systems, methods and processes of the present invention will be apparent from the detailed description which follows.

In the examples of the preferred embodiments the ingredients are combined such that one recommended dose of the composition provides the following approximate quantity of each ingredient:

### EXAMPLE 1

### DHT Blocker Formulation

| | | |
|---|---|---|
| Zinc | 0-20 | mg |
| Selenium | 0-200 | μg |
| Polygonum multiflorum | 0-200 | mg |
| Saw Palmetto berry extract | 1-550 | mg |
| Pumpkin seed extract | | |
| beta sitosterol and/or | | |
| quercetin | | |
| Lycopene: (for example, Tomato extract) | 0-4 | mg |
| Isoflavones: (for example, soybean and/or | 1-40 | mg |
| Pueraria lobata root extract) | | |

### EXAMPLE 2

### DHT Blocker Formulation

| | | |
|---|---|---|
| Zinc | 10 | mg |
| Selenium | 100 | μg |
| Polygonum multiflorum | 100 | mg |
| Saw Palmetto berry extract | 1-275 | mg |
| Pumpkin seed extract | | |
| Quercetin and/or beta sitosterol | | |
| Lycopene (for example, Tomato extract) | 2 | mg |
| Isoflavones: (for example, soybean and/or | 1-20 | mg |
| Pueraria lobata root extract) | | |

In a preferred embodiment the above-described formulations are put in a tablet form to administer daily to an individual for the prevention or treatment of the detrimental effects of DHT.

References

The following references are incorporated herein in their entirety.
1. Bach D, Ebeling L. Long-term drug treatment of benign prostatic hyperplasia-results of a prospective 3-year multicenter study using Sabal extract IDS 89. Phytomed. 1996; 3:105-11.
2. Carraro J C, Raynaud J P, Koch G, et al. Comparison of phytotherapy (Permixon®) with finasteride in the treatment of benign prostate hyperplasia: A randomized international study of 1,098 patients. Prostate. 1996; 29:231-40.
3. Carbin B E, Larsson B, Lindahl O. Treatment of benign prostatic hyperplasia with phytosterols. Br J Urol. 1990; 66:639-41.
4. Carbin B E, Eliasson R. Treatment by Curbicin in benign prostatic hyperplasia (BPH). Swed J Biol Med. 1989; 2:7-9.
5. Schiebel-Schlosser G, Friederich M. Phytotherapy of BPH with pumpkin seeds—a multicenter clinical trial. Zeits Phytother. 1998; 19:71-6.
6. Meydani M. Biol trace Elem Res. 1995; 33:79-86.
7. Clark L C, Combs G F, Turnbull B W, et al. Effects of selenium supplementation for cancer prevention in patients with carcinoma of the skin. JAMA. 1996; 276(24):1957-1963.
8. Clark L C, Dalkin B, et al. Decreased incidence of prostrate cancer with selenium supplementation: result of a double-blind cancer prevention trial. Brit J Urol. 1998; 81:730-734.
9. Yoshizawa K, Willett W C, Morris S J, et al. Study of prediagnostic selenium levels in toenails and the risk of advanced prostate cancer. J Natl Cancer Inst 1998; 90:1219-24.

What is claimed is:

1. A composition for the prevention or treatment of the detrimental effects of dihydrotestosterone comprising Saw Palmetto berry extract, pumpkin seed extract, and at least one of beta-sitosterol, and quercetin.

2. The composition of claim 1, wherein said composition further comprises at least one member of the group consisting of Polygonum multiflorum root extract, lycopenes, tomato extract, isoflavones, Pueraria lobata root extract, soybean extract, a mineral, a pharmaceutically acceptable carrier, an excipient, a preservative, and any combination thereof.

3. The composition of claim 2, wherein said mineral component comprises a zinc-containing component, a selenium-containing component or a combination thereof.

4. The composition of claim 3, wherein said zinc-containing component is zinc arginate.

5. The composition of claim 3, wherein said selenium-containing component is selenomethionine.

6. The composition of claim 2, wherein the composition comprises from about 0.1% to about 80% of Saw Palmetto berry extract, from about 0. 1% to about 80% pumpkin seed extract, from about 0% to about 80% beta-sitosterol, from about 0% to about 80% quercetin, from about 0.1% to about 20% of zinc arginate, from about 0.01% to about 5% of selenomethionine, from about 0.1% to about 50% of Polygonum multiflorum, from about 0.1% to about 10% of lycopene, from about 0.1% to about 20% of isoflavones.

7. The composition of claim 6, wherein the composition comprises from about 10% to about 70% of Saw Palmetto berry extract, from about 10% to about 70% pumpkin seed extract, from about 0% to about 70% beta-sitosterol, from about 0% to about 70% quercetin, from about 1% to about 10% of zinc arginate, from about 0.015% to about 2% of selenomethionine, from about 15% to about 35% of Polygonum multiflorum, from about 0.5% to about 1% of lycopene, from about 1% to about 10% of isoflavones.

8. A method for the prevention or treatment of the detrimental effects of dihydrotestosterone comprising the steps of:
providing a composition comprising Saw Palmetto berry extract, pumpkin seed extract, and at least one of beta-sitosterol, and quercetin; and
administering an effective amount of said composition to an individual in need thereof, in a pharmaceutically acceptable form.

9. The method of claim 8, wherein said composition further comprises at least one of Polygonum multiflorum root extract, lycopenes, tomato extract, isoflavones, Pueraria lobata root extract, soybean extract, a zinc-containing component, zinc arginate, a selenium-containing component, selenomethionine, a pharmaceutically acceptable carrier, an excipient, a preservative or any combination thereof.

10. The method of claim 8, wherein said pharmaceutically acceptable form is chosen from the group consisting of a gel, a lotion, a shampoo, a spray, a powder, a pill, a tablet, an emulsion, a liquid, a salt, a paste, a jelly, an aerosol, an ointment, a capsule, a gel cap, a control release capsule, and any combination thereof.

11. A method for the treatment of hair loss or prostate health comprising the steps of:
providing a composition comprising Saw Palmetto berry extract, pumpkin seed extract, and at least one of beta-sitosterol, and quercetin; and
administering an effective amount of said composition to an individual in need thereof, in a pharmaceutically acceptable form.

12. The method of claim 11, wherein said composition further comprises at least one of Polygonum multiflorum root extract, lycopenes, tomato extract, isoflavones, Pueraria lobata root extract, soybean extract or any combination thereof.

13. The method of claim 11, wherein said composition further comprises at least one mineral component.

14. The method of claim 13, wherein said mineral component comprises at least one of a zinc-containing component, and a selenium-containing component.

15. The method of claim 14, wherein said zinc-containing component is zinc arginate.

16. The method of claim 14, wherein said selenium-containing component is selenomethionine.

17. A composition for treating or preventing the detrimental effects of dihydrotestosterone comprising from about 0 mg to about 550 mg of Saw Palmetto berry extract, from about 0 mg to about 550 mg of Pumpkin seed extract, from about 0 mg to about 550 mg beta sitosterol, from about 0 mg to about 550 mg of quercetin, from about 0 mg to about 20 mg of a zinc-containing component, from about 0 μg to about 200 μg of a selenium-containing component, from about 0 mg to about 200 mg Polygonum multiflorum, from about 0 mg to about 4 mg of a lycopene, and from about 1 mg to about 40 mg of an isoflavone.

18. The composition of claim 17, wherein said lycopene comprises a tomato extract.

19. The composition of claim 17, wherein said isoflavone comprises at least one of a soybean or a Pueraria lobata root extract.
